(19) United States
(12) Patent Application Publication
(10) Pub. No.: US 2026/0259202 A1
(43) Pub. Date: Sep. 3, 2026

(54) COMPOSITIONS AND METHODS FOR THREE-DIMENSIONAL SPATIAL BIOMOLECULE IDENTITY AND ABUNDANCE ASSESSMENT

(71) Applicant: THE REGENTS OF THE UNIVERSITY OF CALIFORNIA, Oakland, CA (US)

(72) Inventors: Xiaoyu Shi, Irvine, CA (US); Elliot En-Yu Hui, Irvine, CA (US)

(21) Appl. No.: 18/994,774

(22) PCT Filed: Aug. 28, 2023
(86) PCT No.: PCT/US23/73024

§ 371 Date: Jan. 15, 2025

Related U.S. Application Data
(60) Provisional application No. 63/373,600, filed on Aug. 26, 2022.

(51) Int. Cl. G01N 33/543; C12Q 1/6841; G01N 33/68

(52) U.S. Cl. CPC G01N 33/5436; C12Q 1/6841; G01N 33/6803

(57) ABSTRACT

Proper functioning of the human body relies on the organization of cells in 3D space. A cell's function and fate are determined by its biomolecule composition and 3D environment. The spatial identification of proteins, RNAs, DNAs, and other biomolecules in a tissue thus provides a powerful map to decipher how cells build tissues and become diseased. Thus, described herein are methods and compositions for assessing biomolecule identity (the present invention features methods and compositions for assessing biomolecule identity (e.g., genes (DNA, RNA), DNA and RNA modifications, proteins, protein modifications, lipids, sugars, metals), and abundance (e.g., RNA expression levels, protein translation levels) in a 3D tissue sample.

### CROSS-REFERENCES TO RELATED APPLICATIONS

This application claims benefit of U.S. Provisional Application No. 63/373,600 filed Aug. 26, 2022, the specification of which is incorporated herein in their entirety by reference.

### STATEMENT REGARDING FEDERALLY SPONSORED RESEARCH OR DEVELOPMENT

This invention was made with government support under Grant No. DP2GM150017 awarded by National Institutions of Health. The government has certain rights in the invention.

### FIELD OF THE INVENTION

The present invention features methods and compositions that utilize photons to spatially release biomolecules from a 3D tissue sample. In particular, the present invention may feature methods and compositions for assessing biomolecule identity (e.g., genes (DNA, RNA), DNA and RNA modifications, proteins, protein modifications, lipids, sugars, metals) and abundance (e.g., RNA expression levels, protein translation levels) in a 3D tissue sample.

### BACKGROUND OF THE INVENTION

Proper functioning of the human body relies on the organization of cells in 3D space. Its biomolecule composition and 3D environment determine a cell's function and fate. The spatial identification of proteins, RNAs, and DNAs in a tissue thus provides a powerful map to decipher how cells build tissues and become diseased. Through single-cell omics, it's been possible to reveal rare cell types that benchmark development, oncogenesis, and brain functions. Multiomics has further transformed the understanding of cancer development by uncovering the genotype-phenotype relationship and gene regulatory network across DNA, RNA, protein, and metabolite. However, the cell isolation process in single-cell analysis unavoidably causes a loss of spatial information. To obtain spatial information, spatial transcriptomics based on imaging or sequencing has emerged to give insight into the heterogeneous expression patterns in tumors, brain, and wound tissues. Unfortunately, most spatial transcriptomics methods can only examine thin tissue sections. Although a 3D model can be reconstructed from a set of 2D images of consecutive sections, this approach is laborious and prone to errors caused by cross-experiment variability, material loss, and distortion by sectioning. Moreover, a shared problem of current spatial transcriptomics methods is their incompatibility with proteomics, which is directly related to the phenotype of a cell.

Emerging spatial omics technologies have transformed the understanding of the spatial expression patterns of biomarkers in tumors, brain, and wound tissues. Five key capabilities are important for evaluating spatial omics methods: spatial resolution, coverage, 3D tissue detection, multiomics, and throughput (FIG. 7A). FIG. 7B summarizes the performance of the current spatial transcriptomics methods and the present invention (i.e., GO3D) across these five features. Imaging-based transcriptomics methods, such as MERFISH and seqFISH+, offer subcellular spatial resolution but lower throughput than sequencing-based methods. On the contrary, sequencing-based methods, such as Spatial Transcriptomics, have much higher throughput but lower spatial resolution. Recently, Slide-seq v219 and High-Definition Spatial Transcriptomics achieved both high throughput and cellular resolution. However, they are incompatible with proteomics, a shared problem for most spatial transcriptomics methods. Multiomics methods, such as laser capture microdissection (LCM) and Digital Spatial Profiler (DSP), could profile target proteins and RNA of the same sample. However, they are limited by another shared problem that most spatial omics methods require thin tissue sections. In summary, it is incredibly challenging to achieve all five key single-cell analysis capabilities simultaneously.

Thus, the present invention (i.e., GO3D technology) features methods and compositions to overcome the disadvantages of the current spatial omics technologies and enables the profiling of proteins, RNAs, DNAs, and other biomolecules of whole-mount tissues in 3D with subcellular resolution, high coverage, and high throughput, simultaneously.

### BRIEF SUMMARY OF THE INVENTION

It is an objective of the present invention to provide compositions and methods that allow for releasing and/or profiling biomolecules in whole-mount tissues in 3D with subcellular resolution, high coverage, and high throughput, simultaneously, as specified in the independent claims. Embodiments of the invention are given in the dependent claims. Embodiments of the present invention can be freely combined with each other if they are not mutually exclusive.

The compositions and methods described herein will drastically transform the understanding of biological questions related to cell organization in 3D, which lacks the proper tools to address currently. For example, how do cells in highly dynamic skin migrate in 3D to heal wounds? How do specialized neurons build the brain? And where do microbes interact with what cell types are in the gut? Thus, the GO3D spatial multiomics technology described herein may be used to address these essential biomedical questions. The answers to these questions are directly relevant to human health and will provide new targets and novel mechanisms for diagnosis and therapeutics.

In some embodiments, the present invention features compositions and methods for assessing biomolecule identity (e.g., genes (DNA, RNA), DNA and RNA modifications, proteins, protein modifications, lipids, sugars, metals) and abundance (e.g., RNA expression levels, protein translation levels) in a tissue sample, which provide deep biomolecule-identifying sequence coverage or mass spectrometry analysis coverage at high-resolution in 3-dimensional space across multiple locations assessed within a tissue sample. The technology described herein closes a significant gap in single-cell analysis that few methods can spatially profile nucleic acids and proteins of whole-mount tissue with subcellular resolution. The ability to measure whole-mount tissues increases the throughput and accuracy of 3D spatial omics by avoiding tissue sectioning. The subcellular resolution assures the precise separation of cells entangled with each other, such as neurons in brains. Multiomics (i.e., detecting more than one type of biomolecules) allows interrogation of the 3D spatial patterns of gene expression, phenotype, genotype, and epigenomics of the same tissue and, therefore, discover the correlated gene regulatory networks. The multiomics information this technology provides will delineate the spatial heterogeneity of tissues responsible for functioning organs, such as the brain, and propagation of diseases, such as cancers and Alzheimer's disease.

In some embodiments, the present invention may also feature novel multifunctional crosslinkers (i.e., gel-based optical isolation (GO3D) crosslinkers) to modify biomolecules (e.g., DNA, RNA, protein) in a tissue sample. The multiple functions in each type of GO3D crosslinker enable gel-based optical cell isolation. Each GO3D crosslinker may comprise a connector that covalently labels biomolecules (e.g., proteins or nucleic acids), a photocleavable anchor that crosslinks labeled biomolecules to the hydrogel, and a reporter, which enables imaging of the crosslinker. In some embodiments, the crosslinkers may further comprise an enrichment handle that can be used to concentrate biomolecules through beads or substrates.

In another aspect, the present invention features methods for obtaining 3D spatially-resolvable biomolecule identity and abundance data from a tissue sample comprising (a) obtaining a tissue sample from a subject, (b) preparing a cryosection of the tissue sample, (c) chemically modify the tissue sample with GO3D multifunctional linkers described herein, (d) forming in situ hydrogels in the tissue sample; (e) releasing biomolecules from single cells in the tissue sample in 3D using lasers or chemicals, where optical cell isolation provides up to 200 nm resolution; (f) capturing released biomolecules and barcoding with DNA oligos and/or isobaric mass spectrometry tags of unique sequences in the capture microfluidics system and (g) obtaining the sequences or protein identities of a population of biomolecules, thereby obtaining 3D spatially-resolvable biomolecule abundance data from the tissue sample.

One of the unique and inventive technical features of the present invention is the use of a novel gel-based optical isolation (GO3D) crosslinker. Without wishing to limit the invention to any theory or mechanism, it is believed that the technical feature of the present invention advantageously provides for the ability to release biomolecules spatially (e.g., proteins, RNAs, DNAs, carbohydrates, or metal-containing enzymes) of whole-mount tissues with subcellular resolution, high coverage, and high throughput. Specifically, the present invention has the following key technologies: cleavable multifunctional crosslinker, multiphoton cell isolation, hydrogel-tissue crosslinking, microfluidics capture and barcoding, and integration with sequencer and mass spectrometer (MS) detection. The workflow of GO3D multiomics is shown in FIG. 1. None of the presently known prior references or work has the unique, inventive technical feature of the present invention.

Furthermore, the prior references teach away from the present invention. For example, most prior references have either high throughput or cellular resolution, and technologies comprising both are incompatible with proteomics. Another shared problem is that most spatial omics methods require thin tissue sections and do not work on whole-mount thick tissues.

Furthermore, the inventive technical features of the present invention contributed to a surprising result. For example, the present invention allows for the precise spatial release of biological molecules in a 3D structure (e.g., a whole-mount tissue sample).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a non-limiting example of a workflow of GO3D spatial multiomics technology described herein.

FIGS. 2A, 2B, 2C, and 2D show a schematic of multifunctional GO3D crosslinkers described herein (FIG. 2A), and non-limiting examples of GO3D crosslinkers synthesized for protein (FIG. 2B), DNA (FIG. 2C), and mRNA (FIG. 2D) profiling.

FIGS. 3A and 3B show in a hydrogel, two-photon cleavage of PXL1 at 730 nm (FIG. 3A) has higher resolution in x, y, and z and is deeper and more efficient, compared with one-photon cleavage at 405 nm (FIG. 3B). Scale bars 100 μm.

FIGS. 4A and 4B demonstrate the subcellular resolution of GO3D optical isolation in a hydrogel. FIG. 4A shows a mixture of cells expressing mCherry or GFP embedded in a hydrogel. FIG. 4B shows after GO3D cell isolation and protein extraction from cells expressing mCherry, only GFP cells are left in the gel (FIG. 4B). Scale bars 10 μm.

FIG. 5 shows a PEG of all free proteins eluted from cell-gel samples and shows high protein-hydrogel crosslinking efficiency. Coomassie Brilliant Blue stain.

FIGS. 6A, 6B, and 6C show the design and prototype of the barcoding microvalve system for the GO3D platform. FIG. 6A shows photocleaved biomolecules drawn into chips by electrophoresis and dispensed into a nanoliter droplet. FIG. 6B shows DNA oligos dispensed directly into droplets, allowing DNA barcode building blocks to be added to captured biomolecules. The inset is a photo of a nanodispenser injecting oligo into a droplet. FIG. 6C shows a 64-nanodispenser chip, which can encode 260,000 unique barcodes.

FIGS. 7A and 7B show a graph comparing with current spatial omics methods; GO3D is expected to have outstanding performance in all five features.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included.

The present invention (e.g., gel-based optical isolation (GO3D)) will close a significant gap in single-cell analysis that few methods can spatially profile nucleic acids and proteins of whole-mount tissue with subcellular resolution. The ability to measure whole-mount tissues will increase the throughput and accuracy of 3D spatial omics by avoiding tissue sectioning. The subcellular resolution assures the precise separation of cells entangled with each other, such as neurons. Multiomics will allow interrogation of the 3D spatial patterns of gene expression, phenotype, genotype, and epigenomics of the same tissue and, therefore, lead to finding the correlated gene regulatory networks. The multiomics information provided by GO3D will delineate the spatial heterogeneity of tissues, which is responsible for the functioning of organs, such as skin and brain, and the propagation of diseases, such as cancers and Alzheimer's disease.

The present invention has the potential for a comprehensive impact on biomedical research. The GO3D multiomics technology will be an ideal tool to study the heterogeneity in tumors, the brain, and many other organs. Tumor heterogeneity provides the fuel for treatment resistance. Therefore, an accurate assessment of tumor heterogeneity that GO3D spatial multiomics can provide is essential for developing effective cancer therapies. In neuroscience, neuron isolation is a big obstacle to single-neuron analysis because using traditional cell isolation methods to dissect single neurons with long projections is challenging. GO3D will remove this obstacle with its 200 nm-resolution optical isolation, which enables precise single-cell dissection across brain regions. Using the GO3D platform, multiomics profiling of completely intact neurons will be possible for the first time.

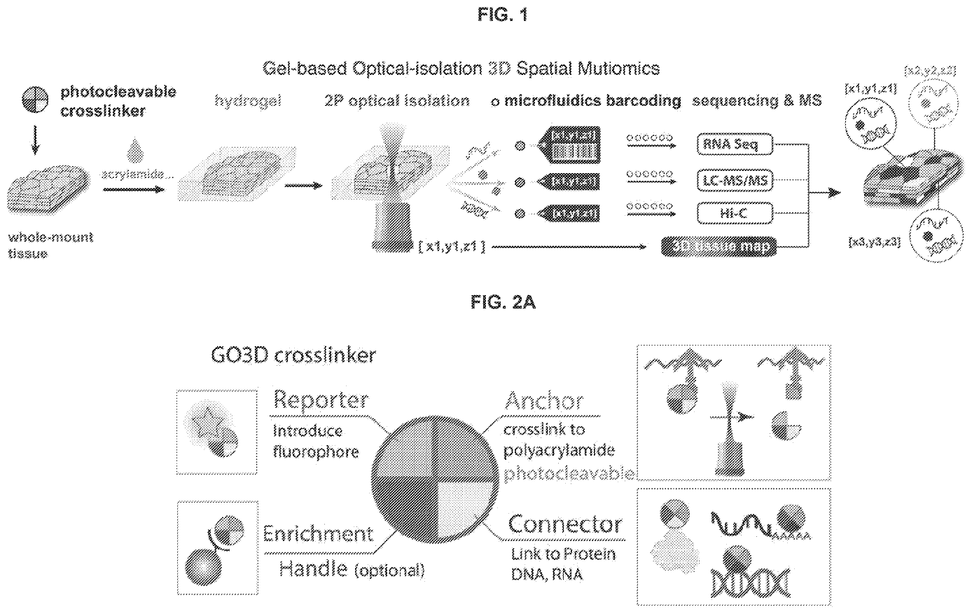

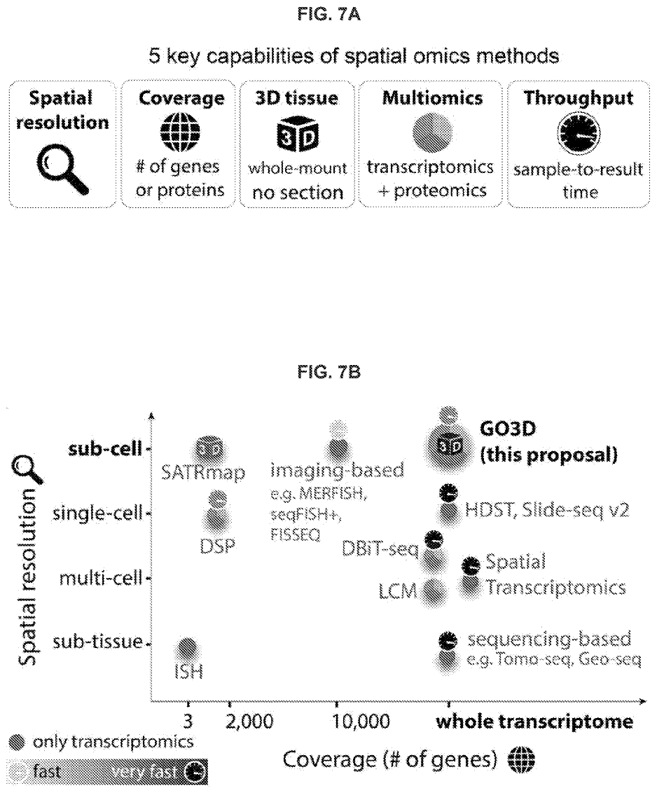

Referring now to FIGS. 1-7B, the present invention features methods and compositions for assessing biomolecule identity (e.g., genes (DNA, RNA), DNA and RNA modifications, proteins, protein modifications, lipids, sugars, metals), and abundance (e.g., RNA expression levels, protein translation levels) in a 3D tissue sample.

### Multifunctional Crosslinkers

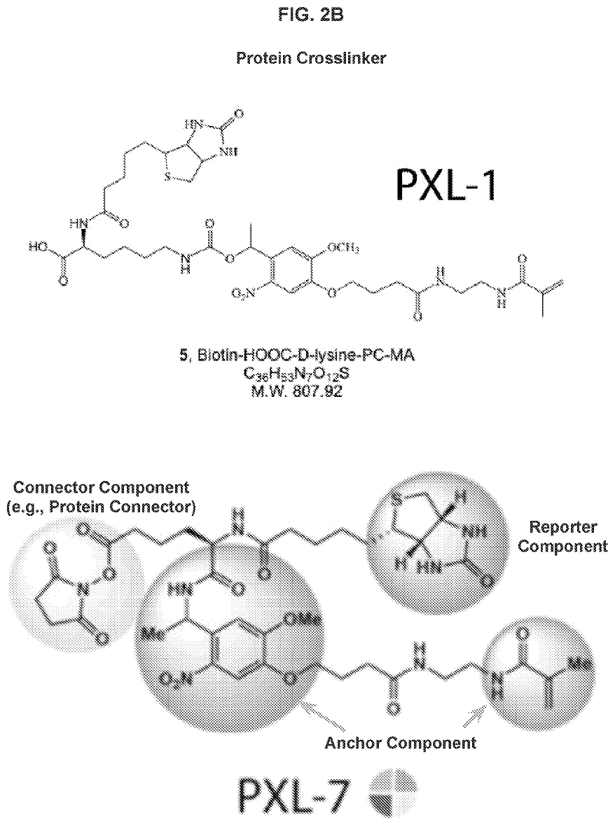

The present invention features novel multifunctional crosslinkers (e.g., gel-based optical isolation (GO3D) multifunctional crosslinkers) to modify biomolecules (e.g., DNA, RNA, protein) in a 3D tissue sample. The multiple functions in each type of GO3D crosslinker enable gel-based optical cell isolation. Each GO3D crosslinker comprises a connector that covalently labels biomolecules (e.g., proteins or nucleic acids), a photocleavable anchor, and a reporter (FIG. 2A-2D). The crosslinker may further comprise an enrichment handle (FIG. 2A-2D).

In some embodiments, the GO3D crosslinker comprises a connector that covalently labels biomolecules (e.g., proteins or nucleic acids), a photocleavable anchor, a reporter, and an enrichment handle (FIG. 2A-2D).

Referring to FIG. 2A-2D, the GO3D multifunctional crosslinkers comprise at least three components connected to each other. For example, each of the GO3D multifunctional crosslinkers comprise a connector component that covalently labels biomolecules (e.g., proteins (FIG. 2B), nucleic acids (FIG. 2C), and RNAs (FIG. 2D)), an anchor component, and a reporter component connected to each other. In some cases, the GO3D multifunctional crosslinkers comprise at least four components each connected to each other (FIG. 2A). For example, each of the GO3D multifunctional crosslinkers comprise a connector component that covalently labels biomolecules (e.g., proteins, nucleic acids, and RNAs), an anchor component, a reporter component, and an enrichment component connected to each other.

As used herein, “biomolecules” refers to organic molecules produced by and essential to a living organism. In some embodiments, the biomolecule is RNA, DNA, protein, lipids, carbohydrates, or metal-containing enzymes. Optionally, the RNA is a poly-A-tailed RNA (e.g., mRNA). In some embodiments, the DNA is genomic DNA or barcode DNA.

In some embodiments, the biomolecules are barcoded with DNA oligonucleotides, protein peptides, fluorophores, or a combination thereof.

The connector refers to GO3D crosslinkers for different biomolecules. For example, in one embodiment, the connector (e.g., a biomolecule-specific crosslinker) is a protein crosslinker (PXL), which may comprise an N-Hydroxysuccinimide (NHS) ester or other chemical groups, including but not limited to maleimide, phenol, carbenes, and cyclopropanones, that react to amino acids in all proteins (see FIG. 2B). The protein crosslinker may comprise an amino-acid-reactive molecule of sufficient length to allow for the capture of proteins. In some embodiments, a DNA barcode may be used as a connector to capture an attached protein. Optionally, the DNA barcode attached to a target protein is an antibody. In some embodiments, the antibody is specifically bound to a target protein; optionally, the antibody-bound target protein possesses a label. In some embodiments, isobaric mass spectrometry tags are used to barcode an attached protein.

In some embodiments, the connector (e.g., a biomolecule-specific crosslinker) is a nucleic acid crosslinker (NXL), which may comprise an alkylating group that reacts to guanine (FIG. 2C). Other examples of a nucleic acid crosslinker include a nucleic acid oligo, formaldehyde, and nucleic acid that can be metabolically labeled to DNAs and RNAs crosslinker under DNA polymerase and RNA polymerase. In other embodiments, the connector (e.g., a biomolecule-specific crosslinker) is an mRNA crosslinker (RXL) and may comprise a poly-thymine that hybridizes the poly-A tail of mRNA (FIG. 2D). The poly-dT tail is of sufficient length to allow for the capture of poly-A-tailed RNAs via hybridization.

In some embodiments, the connector is a lipid crosslinker. In some embodiments, the connector is a carbohydrate crosslinker. In some embodiments, the connector is a metal crosslinker.

In some embodiments, the aforementioned crosslinkers (e.g., nucleic acid, lipid, carbohydrate, or metal crosslinkers) can be used simultaneously for 3D spatial multiomics or individually for one type of omics.

In some embodiments, the photocleavable anchor crosslinks labeled biomolecules to a hydrogel. A photocleavable group will be dissociated into two or more parts when exposed to light. Examples of photocleavable groups include but are not limited to nitrobenzyl derivatives, coumarin derivatives, and caged compounds. In some embodiments, the photocleavable group of the anchor refers to GO3D crosslinkers for different biomolecules. Different photocleavable groups can be photocleaved by photons at different wavelengths.

In some embodiments, the reporter enables imaging of the GO3D multifunctional crosslinker. A reporter can be fluorescent or can be combined with a fluorescent molecule or a fluorescently conjugated molecule. The reporter may comprise biotin, azide or derivatives thereof, alkyne or derivatives thereof, digoxin derivatives, organic dye, triple hemagglutinin tag, histidine tag, nucleic acid oligomer, or a combination thereof.

Without wishing to limit the present invention to any theories or mechanisms, an enrichment handle may be utilized if the released biomolecules are not detected on known laboratory techniques used to measure biomolecule concentration (e.g., mass spectrometry or western blot). In some embodiments, the enrichment handle may comprise biotin, azide or its derivatives, alkyne or its derivatives, digoxin derivatives, organic dye, triple hemagglutinin tag, histidine tag, nucleic acid oligomer, or a combination thereof. The enrichment handle should be able to facilitate concentrating biomolecules to a substrate, such as protein/DNA/RNA purification columns.

In some embodiments, the photocleavable anchor, reporter, and enrichment handle are the same for all GO3D crosslinkers regardless of the target biomolecule.

Many alternative chemical groups may be used to serve the same functions of the GO3D multifunctional linkers described herein. For example, if higher protein labeling efficiency is needed, an additional PXL with a maleimide connector can be used together with the NHS ester PXL. Maleimide-thiol reaction is widely used to produce protein-polymer conjugates for therapeutics. If the polyacrylamide hydrogel may retain some biomolecules during their release, other polymers may be used with less non-specific binding to biomolecules, such as zwitterionic hydrogels.

### Methods

The present invention also features methods for assessing biomolecule identity (e.g., genes (DNA, RNA), DNA and RNA modifications, proteins, protein modifications, lipids, sugars, metals), and abundance (e.g., RNA expression levels, protein translation levels) in a 3D tissue sample.

The present invention features a method for releasing biomolecules from a 3D tissue sample. The method may comprise chemically modifying biomolecules in a 3D tissue sample with multifunctional crosslinkers as described herein (e.g., the multifunction crosslinker comprises a connector to covalently label a biomolecule, a photocleavable anchor, and a reporter), forming in situ hydrogels within the 3D tissue sample using the multifunctional crosslinkers to hold biomolecules in their original position within the 3D tissue sample, and releasing the biomolecules from a portion of the 3D tissue sample. The method may further comprise capturing and barcoding the released biomolecules in a capture microfluidics system. In some embodiments, the method may further comprise capturing, enriching, and barcoding the released biomolecules. The method may also comprise obtaining sequence or protein identities of a population of the released biomolecules.

The present invention may also feature a method for obtaining 3D spatially-resolvable biomolecule identity and abundance data from a 3D tissue sample. The method may comprise chemically modifying biomolecules in a 3D tissue sample with multifunctional crosslinkers, as described herein (e.g., the multifunction crosslinker comprises a connector to covalently label a biomolecule, a photocleavable anchor, and a reporter). Next, in situ hydrogels are formed within the 3D tissue sample using the multifunctional crosslinkers to hold biomolecules in their original position within the tissue sample, and then the biomolecules are released from a portion of the 3D tissue sample. The method may additionally comprise capturing released biomolecules and barcoding with DNA oligos and/or isobaric mass spectrometry tags of unique sequences in the capture microfluidics system and obtaining the sequences or protein identities of a population of the released biomolecules. Thereby obtaining the 3D spatially-resolvable biomolecule abundance data from the tissue sample.

In some embodiments, the method may comprise (a) obtaining a 3D tissue sample from a subject, (b) preparing a cryosection of the 3D tissue sample, (c) chemically modifying the 3D tissue sample with a multifunctional crosslinker (e.g., GO3D multifunctional crosslinkers described herein), (d) forming in situ hydrogels in the tissue sample, (e) releasing the biomolecules from a portion of the 3D tissue sample, (f) capturing released biomolecules and barcoding with DNA oligos of unique sequences in the capture microfluidics system and (g) obtaining the sequences or protein identities of a population of biomolecules. Thus, obtaining 3D spatially-resolvable biomolecule abundance data from the tissue sample.

The present invention may also feature a method for obtaining 3D spatially-resolvable biomolecule identity and abundance data from a 3D tissue sample. The method may comprise chemically modifying biomolecules in a 3D tissue sample with multifunctional crosslinkers as described herein, forming an in situ hydrogel within the 3D tissue sample using the multifunctional crosslinkers to hold biomolecules in their original position within the tissue sample, releasing the biomolecules from a portion of the 3D tissue sample, and transporting the biomolecules by applying an electric field. In some embodiments, each of the multifunctional crosslinkers comprises a connector, wherein the connector covalently labels the biomolecules, a photocleavable anchor, and a reporter. In other embodiments, each of the multifunctional crosslinkers comprises a connector, wherein the connector covalently labels the biomolecules, a photocleavable anchor, a reporter, and an enrichment handle. The method may further comprise capturing the released biomolecules in a microfluidics system. In some embodiments, the method further comprises barcoding the released biomolecules in the microfluidics system. In other embodiments, the method further comprises capturing the released biomolecules on a solid substrate. The methods may also comprise obtaining the sequences or protein identities of a population of the released biomolecules, thereby obtaining the 3D spatially-resolvable biomolecule abundance data from the tissue sample.

In some embodiments, chemically modifying biomolecules in a 3D tissue sample with multifunctional crosslinkers comprises covalently labeling the biomolecules with the connector portion of the multifunctional crosslinkers described herein. In some embodiments, the photocleavable anchor portion of the multifunctional crosslinkers described herein holds the biomolecules in their original position within the 3D tissue sample.

In some embodiments, the 3D tissue sample is obtained from a subject. In certain embodiments, the subject is a mammal, optionally a human. The 3D tissue sample may be obtained from the brain, eyes, esophagus, heart, lung, liver, kidney, pancreas, intestine, stomach, tumor, or any organ or body part of the subject. In certain embodiments, the subjects are invertebrates, such as worms or insects. In other embodiments, the subjects are vertebrates other than mammals, such as fish or birds. In certain embodiments, the subject contains or is infected by viruses, bacteria, or parasites, and the tissue sample may comprise intestinal tissue with a microbiome or lung tissue infected by viruses. In certain embodiments, the subject is modified by gene editing or chemical modifications. In some embodiments, the 3D tissue sample is modified by gene editing or chemical modifications. In some embodiments, the tissue sample comprises a single cell. In other embodiments, the tissue sample comprises a whole animal sample. In some embodiments, wherein the whole animal is an invertebrate or a vertebrate. In further embodiments, the tissue sample is cryosectioned. Optionally, the tissue sample is fixed by chemicals, such as formaldehyde and paraformaldehyde. In some embodiments, the tissue sample is fixed with paraffin. In other embodiments, the tissue sample is fixed using formalin fixation and paraffin embedding (FFPE).

In some embodiments, the biomolecule is RNA, DNA, protein, lipids, carbohydrates, metal-containing enzymes, or a combination thereof. Optionally, the RNA is a poly-A-tailed RNA (e.g., mRNA). In some embodiments, the DNA is genomic DNA or barcode DNA. The biomolecules may be barcoded with DNA oligonucleotides, protein peptides, fluorophores, or a combination thereof.

In some embodiments, the biomolecules are released using lasers that cleave the photocleavable anchor to release the biomolecules from the hydrogel. In some embodiments, the biomolecules are released by using light or chemicals or by changing temperature, pH value, or redox conditions. In some embodiments, laser and/or chemicals cleave the photocleavable anchor to release the biomolecules from the hydrogel. Without wishing to limit the present invention to any theories or mechanisms, optical cell isolation is believed to provide up to 200 nm resolution. For example, an anchor containing nitrobenzyle group crosslinks a protein to the polyacrylic hydrogel. Shining laser light at a wavelength that cleaves nitrobenzyle group on the sample can break the nitrobenzyle-containing anchor into two parts. The photocleavage process dissociates the protein from the hydrogel. Therefore, the protein can be released from the hydrogel.

In some embodiments, a portion of the 3D tissue sample comprises a single cell. In other embodiments, a portion of the 3D tissue sample comprises a subcellular region. In further embodiments, a portion of the 3D tissue sample comprises multiple cells.

In some embodiments, the methods described herein further comprise placing the hydrogel onto a solid support. The solid support may comprise an optically transparent substrate. In other embodiments, the solid support may comprise an opaque substrate. In one embodiment, the solid support is a slide. Optionally, the solid support is a glass slide, glass coverslip, plastic, or metal sheet. In some embodiments, the solid support is conductive or coated with conductive material, such as Indium Tin Oxide (ITO) coated glass. In some embodiments, the solid substrate is coated with molecules to capture the enrichment handles of the GO3D crosslinkers. Examples of coating molecules include but are not limited to streptavidin, avidin, biotin, azide or its derivatives, alkyne or its derivatives, anti-digoxin antibodies, anti-hemagglutinin antibodies, other antibodies, Nickel (Ni²⁺), Cobalt (Co²⁺), Copper (Cu²⁺), nucleic acid oligomers, or a combination thereof.

An electric field may be applied to transport released biomolecules to the capture position. In some embodiments, the capture position is a substrate. In other embodiments, the capture position is an entrance to the microfluidic system (e.g., the microfluidic device). In some embodiments, an electric field may be applied to transport released biomolecules to the capture substrate, such as a glass surface or membrane. The electric field may be applied with electrodes formed from materials such as platinum, silver, copper, gold, aluminum, lithium, carbon, or nickel. To avoid electrolysis, low voltages in the range of 1.5 V or below may be employed. Without wishing to limit the present invention to any theory or mechanism, it is believed that using a low voltage avoids electrolysis and the resulting bubbles.

Alternatively, higher voltages up to and beyond 100 V can be employed by structuring the device such that any bubbles that may be formed have a path to be removed from the system instead of obstructing the capture of biomolecules. Bubble removal may be accomplished by providing a path to allow bubbles to float away via buoyancy. Active bubble removal may also be employed, such as by application of vacuum pressure through a semipermeable membrane or by applying liquid flow. Without wishing to limit the present invention to any theory or mechanism, it is believed that the use of high voltage allows for the biomolecules to be captured faster.

In some embodiments, an electric field comprising a voltage of about 1.0 V to 100 V may be applied. In some embodiments, an electric field comprising a voltage of about 0.5 V to 200 V, or about 0.5 V to 150 V, or about 0.5 V to 100 V or about 0.5 V to 75 V, or about 0.5 V to 50 V, or about 0.5 V to 25 V, or about 0.5 V to 10 V, or about 0.5 V to 5.0 V, or about 0.5 V to 2.5 V, or about 0.5 V to 1.5 V may be applied. In other embodiments, an electric field comprising a voltage of about 1.0 V to 200 V, or about 1.0 V to 150 V, or about 1.0 V to 100 V or about 1.0 V to 75 V, or about 1.0 V to 50 V, or about 1.0 V to 25 V, or about 1.0 V to 10 V, or about 1.0 V to 5.0 V, or about 1.0 V to 2.5 V, or about 1.0 V to 1.5 V may be applied. In some embodiments, an electric field comprising a voltage of about 1.5 V to 200 V, or about 1.5 V to 150 V, or about 1.5 V to 100 V or about 1.5 V to 75 V, or about 1.5 V to 50 V, or about 1.5 V to 25 V, or about 1.5 V to 10 V, or about 1.5 V to 5.0 V, or about 1.5 V to 2.5 V may be applied. In some embodiments, an electric field comprising a voltage of about 2.5 V to 200 V, or about 2.5 V to 150 V, or about 2.5 V to 100 V or about 2.5 V to 75 V, or about 2.5 V to 50 V, or about 2.5 V to 25 V, or about 2.5 V to 10 V, or about 2.5 V to 5.0 V may be applied. In other embodiments, an electric field comprising a voltage of about 5.0 V to 200 V, or about 5.0 V to 150 V, or about 5.0 V to 100 V or about 5.0 V to 75 V, or about 5.0 V to 50 V, or about 5.0 V to 25 V, or about 5.0 V to 10 V, or about 10 V to 200 V, or about 10 V to 150 V, or about 10 V to 100 V or about 10 V to 75 V, or about 10 V to 50 V, or about 10 V to 25 V, or about 25 V to 200 V, or about 25 V to 150 V, or about 25 V to 100 V or about 25 V to 75 V, or about 25 V to 50 V may be applied. In some embodiments, an electric field comprising a voltage of about 50 V to 200 V, or about 50 V to 150 V, or about 50 V to 100 V, or about 50 V to 75 V, or about 75 V to 200 V, or about 75 V to 150 V, or about 75 V to 100 V or about 100 V to 200 V, or about 100 V to 150 V, or about 150 V to 200 V may be applied.

In some embodiments, an electric field comprising a voltage of 1.0 V may be applied. In some embodiments, an electric field comprising a voltage of 1.5 V may be applied. In some embodiments, an electric field comprising a voltage of 2.5 V may be applied. In some embodiments, an electric field comprising a voltage of 5.0 V may be applied. In some embodiments, an electric field comprising a voltage of 10 V may be applied. In some embodiments, an electric field comprising a voltage of 25 V may be applied. In some embodiments, an electric field comprising a voltage of 50 V may be applied. In some embodiments, an electric field comprising a voltage of 75 V may be applied. In some embodiments, an electric field comprising a voltage of 100 V may be applied. In some embodiments, an electric field comprising a voltage of 150 V may be applied. In some embodiments, an electric field comprising a voltage of 200 V may be applied. The present invention is not limited to the aforementioned voltages. In some embodiments, an electric field comprising a voltage of less than 1.0 V may be applied. In other embodiments, an electric field comprising a voltage greater than 200 V may be applied.

In some embodiments, the methods described herein may further comprise contacting the hydrogel with a microfluidics system (e.g., a capture and barcoding microfluidics system). A capture material may be used to capture released biomolecules. As used herein, a “capture material” may refer to an emulsion comprising an aqueous phase and an oil phase. In some embodiments, the aqueous phase preserves the functions of the biomolecules captured (i.e., the biomolecules retain proper structure and function).

The methods described herein may be utilized with various microfluidic systems known in the art, such as systems described in PCT/US2018/036327, the disclosure of which is incorporated herein by reference.

In some embodiments, the diameter of channels is 10-1000 microns, the number of dispensers is 64 or any number, and voltage is in a range of 100-1000 V.

TABLE 1
provides specifications established by the GO3D
spatial multiomics platform described herein:

| Resolution | Coverage | 3D detection | multiomics | Throughput |
|---|---|---|---|---|
| 200 nm | transcriptome- | 10 × 10 × 3 mm | transcriptomic, | 86,400 cells |
| | wide, genome-wide, | | proteomics, | per day |
| | proteome-wide | | genomics + Hi-C | |

In some embodiments, the methods described herein may further comprise analyzing the released biomolecules from a portion of the 3D tissue sample using a software. The software may analyze a 3D position of the portion of the 3D tissue sample (e.g., a 3D position of a cell) and/or identity and abundance of the released biomolecules. In some embodiments, the software outputs a multidimensional dataset comprising the 3D position of the portion of the 3D tissue sample (e.g., a 3D position of a cell) and/or the identity and abundance of the released biomolecules.

### Multiphoton Cell Isolation

Multiphoton cell isolation, which is distinct from mechanical or enzymatic homogenization, forms the core component of the GO3D technology described herein. The photocleavage induced by multiphoton laser scanning can trace any shape of cells with ~200 nm resolution, which is precise enough to isolate the finest synapse from its niche. Multiphoton can also penetrate millimeters deep in 3D whole-mount tissues, which allows optical cell isolation in thick tissues. Three-photon (3P) photocleavage can extend the depth of the photocleavage up to 3 mm thick. In GO3D technology, multiphoton is used to spatially release proteins, RNAs, and DNAs after they are crosslinked to hydrogel through photocleavable crosslinkers. The laser is accurately scanned through the single cells, where it delinks biomolecules by breaking up the photocleavable crosslinkers. The laser is also precisely timed to release materials from one cell at a time. This way, the biomolecules from different cells can be separated sequentially and captured individually.

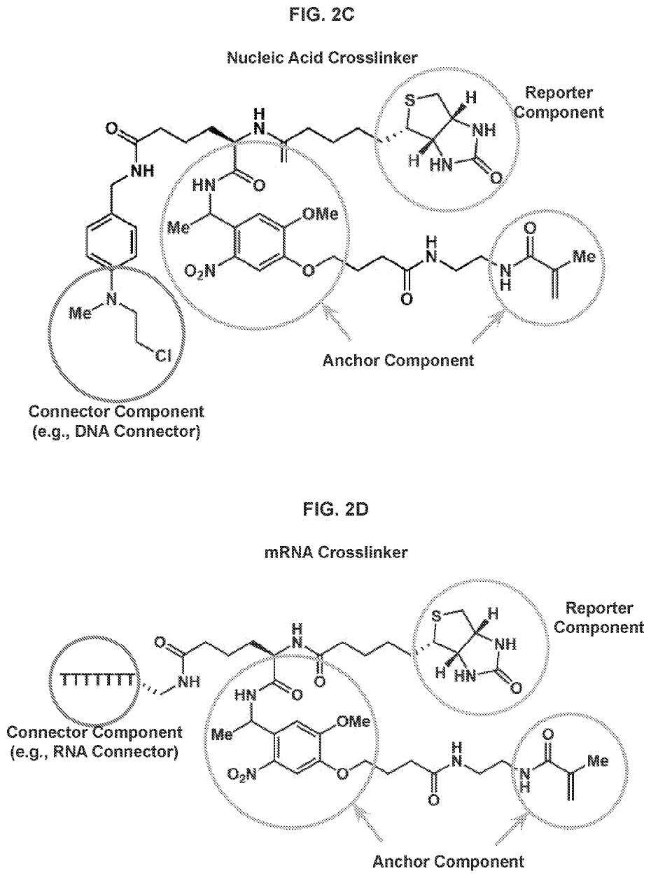

The GO3D resolution is equal to the resolution of photocleavage. Data confirms the 200-nanometer resolution of GO3D using two-photon cleavage in vitro and in situ. First, two-photon cleavage of PXL1 (FIG. 2B) and PXL2 was validated to determine if two-photon cleavage had a higher x, y, and z resolution than one-photon cleavage in 3D hydrogel (FIG. 3A-3B). In some embodiments, PXL1 and PXL2 are both NHS esters, but PXL2 comprises nitrogen instead of oxygen. In some embodiments, PXL2 is more stable than PXL1 for long-term storage. Referring to FIG. 3A-3B, the dark 3D smiley face was created by photocleavage, and the brightness is proportional to the un-cleaved PXL1. The results indicate that two-photon can cleave micrometer-scale patterns like the numbers and big continuous patterns like the smiley face in FIG. 3A. In addition, the higher contrast in FIG. 3A than in FIG. 3B, also reflects that two-photon cleavage is more efficient than one-photon.

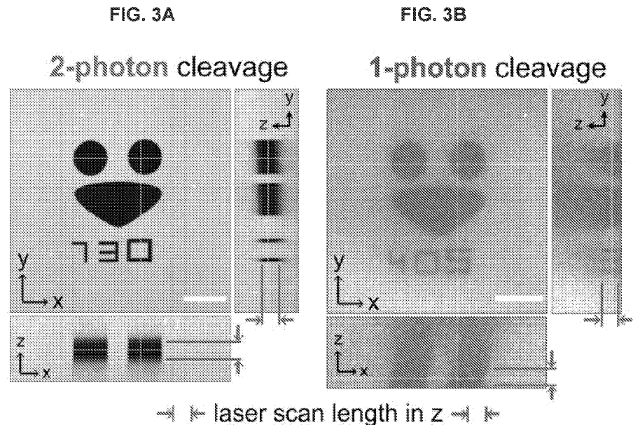

Moving to cells, the subcellular resolution of GO3D optical isolation was confirmed with a mixture of eGFP and mCherry cells embedded in a piece of hydrogel (FIG. 4A-4B). All proteins of all cells are crosslinked to the hydrogel by PXL2, but only the mCherry cells were two-photon cleaved. The post-GO3D image FIG. 4B shows that most proteins in the mCherry cells were cleaved and released, opposite to the eGFP cells. The cleaved cells lost phase contrast in the brightfield channel, demonstrating that the proteins were gone.

The resolution and efficiency of photocleavage in tissue are similar to those in cultured cells. However, the biomolecule released from tissue is sometimes less efficient because extracellular matrix and complex cell-cell interactions may trap proteins in tissues. To break down the nascent protein crosslinking, thorough protein denaturation of the tissue-gel system is performed. For nucleic acid release, DNAse is used to fragment DNA.

Hydrogel Crosslinking with Photocleavable Linkers

In GO3D, the hydrogel functions as a 3D scaffolding to hold biomolecules in their original position in tissues. During the in-situ gel formation, the tissue is permeabilized and crosslinked into the hydrogel as a tissue-gel system through photocleavable GO3D crosslinkers, where optically delinked biomolecules can be freely extracted out of the gel for downstream analysis without disturbing the biomolecules in surrounding cells and extracellular matrix.

Compared with RNA and DNA, the high efficiency of protein-gel anchoring is the most crucial because proteins cannot self-replicate to amplify signals. Low efficiency of either labeling or crosslinking will cause significant protein loss before the optical isolation. However, the present invention features a high crosslinking efficiency between biomolecules (e.g., protein, RNA, DNA) and hydrogel using the GO3D multifunctional crosslinkers described herein.

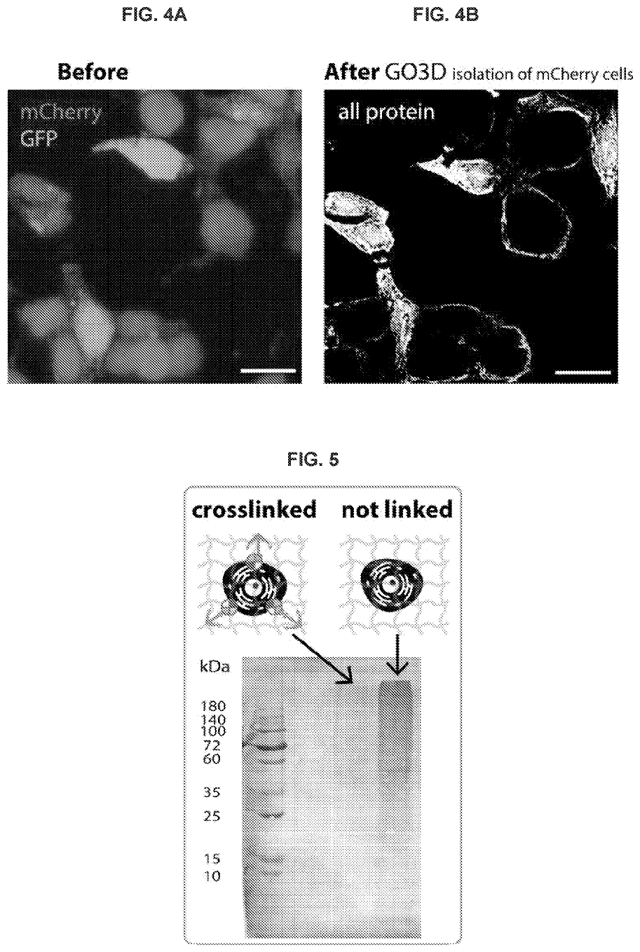

The photocleavable PXL structure described herein is optimized for high protein-gel crosslinking efficiency. A PEG experiment confirmed that the PXL (FIG. 2B) anchored the vast majority of proteins to the hydrogel (FIG. 5). In this experiment, one piece of polyacrylamide hydrogel contains cells crosslinked with PXL2. In contrast, the other gel piece contains untreated cells. After elution and electrophoresis, the sample with PXL2 showed a negligible protein band, which indicates the vast majority of proteins were labeled and crosslinked to the hydrogel by PXL2. The other sample without protein-gel crosslinking showed strong protein bands, which means a significant amount of proteins are lost from the gel.

### MICROFLUIDICS and DNA-Oligo/Isobaric Tag Barcoding System

The capture and barcoding of biomolecules is the last step for GO3D sample preparation. As described herein, microfluidics can capture and barcode single cells, subcellular regions, or multicellular regions with high throughput. The GO3D microfluidics system captures and barcodes sequentially released biomolecules. The barcoded biomolecules are sent to the downstream sequencer and mass spectrometer (MS) for profiling. A part of the GO3D microfluidics system is the DropShop system.

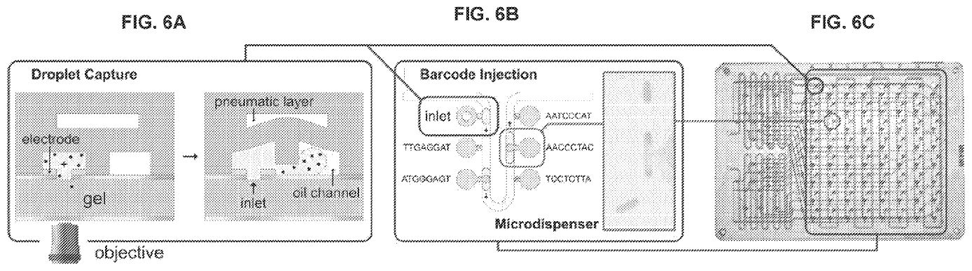

DropShop (FIG. 6B-6C), a microfluidic droplet assembly line, forms the core of the microfluidics capture and barcoding system described herein. Biomolecules released by photocleavage are concentrated by electrophoresis into an inlet port on the chip, which is the rate-limiting step performed at about 1 Hz. The biomolecules will then be drawn into a negative-pressure oil channel by opening a pneumatic valve for about 100 milliseconds (FIG. 6A). The captured droplet then travels along the oil channel past an array of valved nanodispensers, each of which can be triggered to dispense a different DNA oligonucleotide or isobaric mass spectrometry tags into the droplet (FIG. 6B). DNA barcodes will be constructed by assembling six oligo building blocks together head to tail, with eight coding options at each of the six positions, giving a total of 262,144 unique barcodes. This requires 48 unique nanodispensers, which can be accommodated on the 8×8 chip (FIG. 6C). Each barcode is programmed as a function of the timing of biomolecule isolation and therefore records the biomolecule position in the tissue. DropShop employs pneumatic logic circuits to enable the addressing of huge nanodispenser arrays along with computer vision for droplet tracking and synchronized dispenser control. The output from the microfluidics system is used in high-throughput detection pipelines such as sequencers and mass spectrometers.

A sequencer provides higher throughput than imaging-based sequencing by orders of magnitudes (FIG. 1A-1B). Compared to immunofluorescence-based proteomics, MS protein profiling is faster and more quantitative. In contrast to targeted proteomics assisted by antibodies, MS proteomics allows the unbiased discovery of new biomarkers.

### Software for Data Analysis

A new software analyzes the GO3D multiomics data, which includes the 3D position of the cell and the identity and abundance of biomolecules. The barcodes will register all types of biomolecules with the corresponding photocleavage positions of the multiphoton laser. The software will output a multidimensional dataset containing the 3D position of the cell and/or identity and abundance of biomolecules. Using the dataset derived using methods and compositions described herein, one can quantitatively understand tissue heterogeneity, systems regulatory biology, and the genotype-phenotype relationship.

The gel-based optical cell isolation (GO3D) described herein is a conceptually novel concept that is distinct from all cell isolation methods for single-cell analysis. GO3D addresses two shared problems of current spatial transcriptomics methods, which are the incompatibility with thick tissues and proteomics. The GO3D concept is realized through a creative strategy that integrates four technologies: multiphoton cleavage, hydrogel, microfluidics, and sequencing/MS detection. The chemistry foundation of high coverage GO3D profiling of each category of the multiomics is a set of novel photocleavable multifunctional small molecules that efficiently crosslink proteins, RNAs, and DNAs to a hydrogel. Altogether, the integrated GO3D platform allows subcellular, high-coverage, high-throughput, 3D spatial multiomic analysis of whole-mount tissues, which is currently not achievable with existing technologies.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A gel-based optical isolation (GO3D) multifunctional crosslinker comprising:
a) a connector, wherein the connector covalently labels a biomolecule;
b) a photocleavable anchor; and
c) a reporter.

2. The crosslinker of claim 1, further comprising an enrichment handle.

3. The crosslinker of claim 1, wherein the photocleavable anchor crosslinks labeled biomolecules to a hydrogel.

4. The crosslinker of claim 1, where the reporter enables imaging of the crosslinker.

5. The crosslinker of claim 1, wherein the connector is a nucleic acid crosslinker, a protein crosslinker, a lipid crosslinker, a carbohydrate crosslinker, or a metal crosslinker.

6. The crosslinker of claim 5, wherein the nucleic acid connector is an mRNA crosslinker, wherein the mRNA crosslinker comprises a poly-thymine that hybridizes the poly-A tail of mRNA.

7.-8. (canceled)

9. The crosslinker of claim 5, wherein the protein crosslinker comprises an N-Hydroxysuccinimide (NHS) ester or maleimide.

10. (canceled)

11. The crosslinker of claim 1, wherein the biomolecule is RNA, DNA, protein, lipids, carbohydrates, or metal-containing enzymes.

12.-14. (canceled)

15. A method for releasing biomolecules from a 3D tissue sample, the method comprising:
a) chemically modifying biomolecules in a 3D tissue sample with a multifunctional crosslinker according to claim 1;
b) forming an in situ hydrogel within the 3D tissue sample using the multifunctional crosslinker to hold biomolecules in their original position within the 3D tissue sample; and
c) releasing the biomolecules from a portion of the 3D tissue sample.

16. The method of claim 15, further comprising capturing and barcoding the released biomolecules in a microfluidics system.

17. The method of claim 16, further comprising obtaining sequence or protein identities of a population of the released biomolecules.

18. (canceled)

19. The method of claim 15, further comprising
d) transporting the released biomolecules by applying an electric field.

20. The method of claim 19, wherein the electric field is applied with electrodes, wherein the electrodes comprise platinum, silver, copper, gold, aluminum, lithium, carbon, or nickel.

21. (canceled)

22. The method of claim 19, wherein applying the electric field comprises applying a voltage in the range of 1.5 V or below or applying a voltage in the range of 100 V or greater.

23.-30. (canceled)

31. The method of claim 15, wherein the 3D tissue sample is obtained from a subject, wherein the 3D tissue sample is from a brain, eyes, esophagus, heart, lung, liver, kidney, pancreas, intestine, stomach, tumor, or any organ or body part of the subject.

32.-33. (canceled)

34. The method of claim 15, wherein the 3D tissue sample comprises a whole animal sample, wherein the whole animal is an invertebrate or a vertebrate.

35.-38. (canceled)

39. The method of claim 15, wherein the biomolecules are released using lasers or chemicals, wherein the laser or chemicals cleave the photocleavable anchor to release the biomolecules from the hydrogel.

40. The method of claim 15, wherein the portion of the 3D tissue sample comprises a single cell.

41. The method of claim 15, wherein the portion of the 3D tissue sample comprises a subcellular region.

42. The method of claim 15, wherein the portion of the 3D tissue sample comprises multiple cells.

43.-86. (canceled)
